(19) United States
(12) Patent Application Publication
(10) Pub. No.: US 2009/0208553 A1
(43) Pub. Date: Aug. 20, 2009

(54) Monomers and Polymers with Covalently - Attached Active Ingredients

(75) Inventors: Lisa Kay Kemp, Hattiesburg, MS (US); NIcholas Lee Hammond, Hattiesburg, MS (US)

Correspondence Address: HOWREY LLP-HN, C/O IP DOCKETING DEPARTMENT, 2941 FAIRVIEW PARK DRIVE, SUITE 200, FALLS CHURCH, VA, 22042-7195, US

(73) Assignee: University of Southern Mississippi, Hattiesburg, MD (US)

(21) Appl. No.: 12/369,125

(22) Filed: Feb. 11, 2009

Related U.S. Application Data
(60) Provisional application No. 61/066,067, filed on Feb. 15, 2008.

(51) Int. Cl. A61L 27/54; C08L 53/00; A61L 29/16; A61L 31/16

(52) U.S. Cl. 424/423; 525/91

(57) ABSTRACT

Methods to form an active agent modified monomer comprising a ring opening cyclic monomer linked to an active agent via a degradable covalent linkage. Methods to form a polymer or copolymer comprising an active agent modified monomer. Methods to form an active agent modified monomer comprising combining a ring opening cyclic monomer with a first functional group (X) and an active agent with a second functional group (Y) to form an active agent modified monomer, wherein the first (X) and second (Y) functional groups are complementary functional groups that form a degradable linkage. The active agent modified monomer can also comprise a non-degradable linkage. The method can form a ring opening cyclic monomer that includes a cyclic carbonate, cyclic epoxide, lactam, lactone, lactide anhydride, cyclic carbamate, cyclic phosphoester, or siloxane. Apparatus that includes a medical device that comprises a polymer or copolymer that comprises an active agent modified monomer.

### CROSS REFERENCE TO RELATED APPLICATIONS

The present application claims the benefit of U.S. Provisional Patent Application Ser. No. 61/066,067, which was filed on Feb. 15, 2008 and hereby is incorporated by reference in its entirety.

### FEDERALLY SPONSORED RESEARCH

This invention was made with government support under National Science Foundation (NSF) grant #0712489 and NSF grant #0802790. The government may have certain rights in this invention.

### FIELD OF THE INVENTION

The invention relates to monomers and polymers therefrom with covalently-attached active ingredients and, more specifically, to materials and coatings for medical devices and implants.

### BACKGROUND OF THE INVENTION

Coronary heart disease (CHD) is the single largest killer of men and women in the United States today, with over 13.2 million Americans affected; a coronary event occurs every 26 seconds, and a death occurs in the U.S. every 60 seconds from CHD. The leading treatment post event is a drug-eluting stent (DES), and the United States market for DES currently exceeds $5.4 billion. Recent advances in DES technology have increased the success rate of CHD treatment. However, restenosis (reblockage of the artery through the stent) currently occurs in 10 percent of the implanted stents. Also, the drug eluting coatings on the market deliver the bulk of the loaded drug within the first 48 hours of deployment, with little to no delivery after 30 days. Growth of endothelial cells progresses for up to 6 months, thus, there is a need to extend drug delivery by up to 6 months or even longer. Another major problem of late stent thrombosis (blood clotting) exists for the two FDA-approved stents. While these polymer-coated metal stents provide adequate vessel-opening strength during the early days after deployment, they remain stiff for the lifetime of the stent and prevent the natural movement and self-cleaning mechanism of the artery. Repairing restenosis is often an invasive vascular surgery that requires a much longer hospital stay and is much more painful for the patient, and late stent thrombosis can cause the patient to experience a heart-attack, resulting in death for 70 percent of the cases.

There are currently two commercial polymer products for DES coatings. The first consists of a polystyrene-polyisobutylene-polystyrene (PS-PIB-PS) triblock copolymer. While the coating has appropriate physical properties, the drug is not compatible with the polymer system and resides as particles embedded within the polymer matrix. The drug is released within the first 48 hours of implantation and there is no appreciable release after the next 30 days. The second commercial product utilizes a 3 layer system: a primer layer of parylene C onto which is sprayed a solution of two biodegradable polymers, polyethylene-co-vinyl acetate (PEVA) and poly n-butyl ethacrylate (PBMA), containing the drug. The top layer is a drug-free coating of PEVA and PBMA that acts as a diffusion barrier for the drug. These current products each have shown disadvantages with rapid drug release (the drug is incompatible with the polymer matrix and surface clusters of drug show a “burst” release) and have been shown to prevent healing around the stent area leading to thrombosis. Because of the concern over late stent thrombosis, there has been a small shift away from DES and a return to the use of bare-metal stents.

Research has been conducted to evaluate the use of poly(hydroxy styrene)-4, polymethylmethacrylate-5, poly(hydroxymethylmethacrylate)-5, and poly(e-caprolactone)-6 block copolymers with PIB centerblocks as drug release materials. Another group has explored copolymers consisting of poly(butyl acrylate) or poly(lauryl acrylate) soft blocks and hard blocks composed of poly(methyl methacrylate), poly(isobornyl acrylate), or poly/styrene. This work has revealed the applicability of block polymers for use as stent coatings, but no one has examined the combination of properties from microphase separated biostable and biodegradable blocks to achieve a biotransformable stent material that will allow the transport of blood components through the wall of the stent into the vessel wall.

Fully degradable materials for use within stent devices have been studied, but use of the material has been plagued with inflammation issues due to the local concentration of degradation products or with difficulties achieving appropriate physical properties. The most promising of these materials to date is the Reva poly(DTE carbonate) stent with tyrosine-derived polycarbonates, which has shown minimal inflammatory response due to the degradation byproducts. However, human trials are needed. The fully degradable stents create concerns regarding their active agent deliverability and it has yet to be determined if the stents will degrade too quickly in comparison to the rate of healing for the vessel or if the presence of large fragments of the degrading stent will pose a problem.

U.S. Pat. Appl. No. 2007-0020308 to Robert Richard, et al. and U.S. Pat. Appl. No. 2006-0013867 to Robert Richard, et al. describe therapeutic polymers that contain at least one polymeric portion and at least one therapeutic agent. The therapeutic agent and the polymeric portion are covalently linked via one or more linkages that hydrolyze in an aqueous environment, for example, one or more linkages selected from a Si-N linkage, a Si-O linkage, and a combination of the same. Other applications are directed to methods of making the above therapeutic polymers. The applications relate to medical devices that contain isobutylene copolymers. The applications also relate to biocompatible copolymer materials for therapeutic agent delivery comprising a therapeutic-agent-loaded isobutylene copolymer. According to an aspect of the applications, a medical device is provided that includes (a) a substrate and (b) at least one polymeric layer that contains a copolymer disposed over all or a portion of the substrate. The copolymer contains one or more polymer chains within which isobutylene and elevated Tg monomers (and, optionally, other monomers) are incorporated in a random, periodic, statistical or gradient distribution. A polystyrene-random-polyisobutylene copolymer was prepared by using well known cationic polymerization techniques. Permanent bonds are formed by the processes described by these applications.

None of the references solve the stent problems of metal that it is too rigid and releases no active agents or of polymer coatings with embedded active agents that are dispersed too quickly. Some references form a polymer backbone, and then attach a therapeutic agent with a permanent bond. There is a need for gradual active agent delivery from a continuous, relatively homogeneous polymer composition. There is a need to form this composition by utilizing different functional groups by which the attachment of an active ingredient to a polymer backbone can be achieved. Historically, steric hindrance has prevented polymerizing a monomer with an attached therapeutic agent. The references do not designate any material with a therapeutic agent covalent attachment that is an acid or other functional moiety for forming a polymer or copolymer. The references that describe a composition for use as a stent use a material that utilizes a benzyl protecting group for glycerol or no protecting group at all rather than a therapeutic agent.

### SUMMARY

The present invention provides methods to form an active agent modified monomer comprising a ring opening cyclic monomer linked to an active agent via a degradable covalent linkage and methods to form a polymer or copolymer comprising an active agent modified monomer. In an embodiment, the present invention provides methods to form an active agent modified monomer comprising combining a ring opening cyclic monomer with a first functional group (X) and an active agent with a second functional group (Y) to form an active agent modified monomer, wherein the first (X) and second (Y) functional groups are complementary functional groups that form a degradable linkage. The active agent modified monomer can also comprise a non-degradable linkage. In an aspect, the ring opening cyclic monomer can include a cyclic carbonate, cyclic epoxide, lactam, lactone, lactide, anhydride, cyclic carbamate, cyclic phosphoester, or siloxane.

In another embodiment, the present invention provides methods of forming an active agent modified monomer comprising combining a ring opening cyclic carbonate or epoxide monomer with a functional group (L), an active agent with a functional group (Y), and a linker with a functional group (X) and a functional group (M) to form an active agent modified monomer. In an aspect, the functional groups (X) and (Y) are complementary functional groups that form a degradable linkage and the functional groups (L) and (M) are complementary functional groups that form a stable or degradable linkage.

The present invention also provides methods to form an active agent modified monomer comprising a compound including ring-forming complementary groups linked to an active agent via a degradable covalent linkage and methods to form a polymer or copolymer comprising an active agent modified monomer. The present invention provides methods to form an active agent modified monomer comprising combining a compound including a ring-forming complementary group with a first functional group (X) and an active agent with a second functional group (Y) to form an active agent modified monomer, wherein the first (X) and second (Y) functional groups are complementary functional groups that form a degradable linkage. The active agent modified monomer can also comprise a non-degradable linkage. In an aspect, the ring-forming complementary groups can include an alcohol and chloroformate, an alcohol and an acid, an amine and an alcohol, amine and an acid, acid halide and an alcohol, an acid halide and an amine, chloride and alcohol, two alcohols, or two acids.

In another embodiment, the present invention provides methods of forming an active agent modified monomer comprising combining a compound including ring-forming complementary groups with a functional group (L), an active agent with a functional group (Y), and a linker with a functional group (X) and a functional group (M) to form an active agent modified monomer. In an aspect, the functional groups (X) and (Y) are complementary functional groups that form a degradable linkage and the functional groups (L) and (M) are complementary functional groups that form a stable or degradable linkage.

The present invention also provides an apparatus that includes a medical device that comprises a polymer or copolymer that comprises active agent modified monomer units.

### BRIEF DESCRIPTION OF THE FIGURES

FIG. 1 is a schematic representation of the structures formed by an embodiment of the invention.

### DETAILED DESCRIPTION OF THE INVENTION

This invention relates to monomers and polymers formed therefrom with covalently-attached active ingredients. The covalent bonds degrade over time releasing the active ingredients. The present invention also relates to a method for covalent attachment of active agents to the backbone of a degradable polymer by creating active agent-modified monomers that can be polymerized using Ring Opening Polymerization (ROP). The method by which the active agent is converted to a ring opening monomer can vary depending on the functional group present on the active agent.

In another embodiment, the present invention provides methods of forming an active agent modified monomer comprising combining a ring opening cyclic carbonate or epoxide monomer with a functional group (L), an active agent with a functional group (Y), and a linker with a functional group (X) and a functional group (M) to form an active agent modified monomer. In an aspect, the functional groups (X) and (Y) are complementary functional groups that form a degradable linkage and the functional groups (L) and (M) are complementary functional groups that form a stable or degradable linkage.

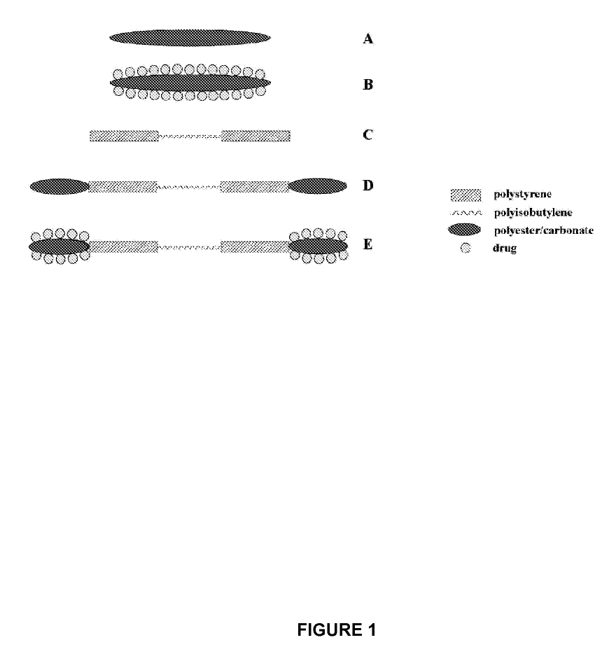

Generally, this invention pertains to ester monomers, carbonate monomers, epoxide monomers, lactam monomers, lactone monomers, lactide monomers, or siloxane monomers, and polymers formed therefrom containing one or more of such units with covalently-attached active ingredients. Homopolymers, copolymer, block copolymer, and higher architectures are synthesized using ROP of cyclic monomers and/or monomers with covalently-attached active ingredients. For example, cyclic carbonates can be synthesized from glycerol, a common trifunctional alcohol, or other alcohols. Naproxen is an exemplary active agent for the following analysis. FIG. 1 provides a simplified comparison of the way these components can be configured.

In embodiments of the present invention, the ring opening cyclic monomer can be a cyclic carbonate, cyclic epoxides, lactam, lactone, lactide, anhydride, cyclic carbamate, cyclic phosphoester, or siloxane. In an aspect, the cyclic epoxide can be glycidol, ethyl-2,3-epoxybutyrate, glycidyl methacrylate, or 1,2,7,8-diepoxyoctane. In other embodiments of the present invention, the cyclic epoxide can be 3,4-Epoxy-1-butene, 2-Methyl-2-vinyloxirane, epichlorohydrin, epibromohydrin, 1,2-epoxy-5-hexene, glycidol propargyl ether, or methyl-2-methylglycidate. The lactam monomer can be 4-Oxo-2-azetidinecarboxylic acid, 4-Hydroxy-2-pyrrolidone, 5-(Hydroxymethyl)-2-pyrrolidinone, Pyroglutamic acid, Ethyl 2-oxo-3-piperidinecarboxylate, or alpha-Amino-epsilon-caprolactam. In some embodiments of the present invention, the lactam can be bromocaprolactam, vinylcaprolactam, 5-chloromethyl-2-pyrrolidinone, 4-(2-propenyl)-2-pyrrolidinone, or 5-iodo-azocan-2-one. The cyclic carbonate can be 5-ethyl-5-(hydroxymethyl)-1,3-dioxan-2-one, 5-hydroxy-1,3-dioxan-2-one, 4-hydroxy-1,3-dioxolan-2-one, 5-methyl-2-oxo-1,3-dioxane-5-carboxylic acid, or 5-ethyl-2-oxo-1,3-dioxane-5-carboxylic acid. In an aspect, the cyclic carbonate can be

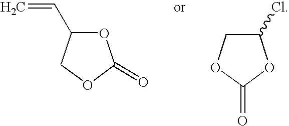

Other suitable ring opening cyclic monomers will be apparent to those of skill in the art and are to be considered within the scope of the present invention.

A polymer produced by ROP of the monomers described herein can be included as an aspect of the present invention. A block-polymer formed by ROP of the monomers described herein and at least one other ring opening monomer using a polymer macroinitiator can also be included as an aspect of the present invention. The monomers described herein can also be polymerized using one or more ring opening monomers to form either a polymer or a block-polymer. Polymers, such as polystyrene, polybutylene, or polyethylene glycol, can be reacted with polymers produced by ROP of monomers described herein to form a block-polymer.

Polymers produced by ROP of the monomers described herein and one or more other ring opening monomers can be used in applications, such as arthritis therapy or in glaucoma therapy. Other suitable applications for these polymers will be apparent to those of skill in the art and are to be considered within the scope of the present invention.

In an aspect, the first (X) or the second (Y) functional group can be independently an amine, aldehyde, ketone, chloroformate, hydrazine, alcohol, carboxylic acid, acid halide, acid anhydride, acid salt, isocyanate, or ester.

In embodiments of the present invention, the active agent can include a non-steroidal anti-inflammatory agents, chemotherapeutic agent, anticoagulant, cholinergics, adrenergics, serotonergics, anesthetics, hypnotics, antiseizure therapeutics, antipsychotics, anxiolytics, stimulants, opiods, analgesics, spasmolytics, cardiac glycosides, antianginals, antiarrhythmics, diuretics, angiotensin converting enzyme inhibitors, angiotensin converting enzyme antagonists, calcium blockers, central sympatholytics, peripheral sympatholytics, vasodilators, antihyperlipoproteinemics, cholesterol biosynthesis inhibitors, antithrombotics, thrombolytics, coagulants, plasma extenders, insulin, oral hypoglycemic agents, adrenocorticoids, estrogens, progestins, androgens, thyroid drugs, nonsteroidial anti-inflammatory agents, antihistamines, antiallergenic agents, antiulcer agents, antibiotics, antimicrobials, antiparasitics, antifungals, antimycobacterial agents, cancer chemotherapeutics, antivirals, protease inhibitors, gene therapeutics, antisense therapeutics, or selective estrogen receptor modulators, carbohydrates, proteins, enzymes, RNA, DNA, pesticides, herbicides, anti-fouling agents, aromatic agents, detergents, sequestering agents, preservatives, anti-corrosion agents, or catalysts.

In some embodiments of the present invention, the functional group (X) or (Y) is an amine, alcohol, carboxylic acid, acid halide, acid anhydride, acid salt, isocyanate, aldehyde, ketone, chloroformate, hydrazine, or ester. In an aspect, the first (X) and second (Y) functional groups can react to form an ester, urethane, anhydride, carbonate, hydrazone, urea, amide bond, or amide degradable linkage.

In other embodiments, the functional group (L) or (M) can be an alkyne, alkene, alkyl halide, azide, thiol, or amine. Other types of compounds that can perform as the functional groups (L), (M), (X), or (Y) will be apparent to those of skill in the art and are to be considered within the scope of the present invention. In another aspect, the functional groups (L) and (M) can react to form a thiolene, triazole, disulfide, or substituted amine.

Embodiments of the present invention can also include a non-degradable covalent linkage (Z) extending between the ring opening cyclic monomer and the degradable covalent linkage. The non-degradable covalent linkage (Z) can include thiolene, triazole, disulfide, or substituted amine. Other suitable compounds that can be used as the non-degradable covalent linkage (Z) will be apparent to those of skill in the art and are to be considered within the scope of the present invention.

Homopolymers. Homopolymers containing ester and carbonate units are synthesized using ROP of commercial polyester monomers and cyclic carbonate monomers that can be modified by covalently attaching a drug. The structures proposed are illustrated schematically in FIG. 1 (A and B) and show both the drug-modified and unmodified polymers.

Block Polymers. Block polymer architectures can be synthesized by using a polymer with appropriate ROP initiating group(s) to polymerize the cyclic monomers. An example of this includes using a combination of quasiliving carbocationic polymerization (QCP) followed by ring-opening polymerization (ROP) of cyclic monomers. The various macromolecular architectures proposed are illustrated schematically in FIG. 1 (C, D, and E). Structure C incorporates the familiar triblock copolymer architecture of classical thermoplastic elastomers, in which high—T_{g} glassy domains provide physical cross linking for low—T_{g} rubbery domains. In addition, structures D and E include a biodegradable block, which provide domains that can be drug modified and become porous after degradation.

### Formation of the Active Agent-Modified Monomer

The active agent can be either linked directly to the ring opening monomer (or precursor to the ring opening monomer) via a degradable bond from reaction with a complementary functional group (Formula 1A and 1C) or it can be attached to a linker molecule via a degradable bond from reaction with a complementary functional group (Formula 1B and 1D).

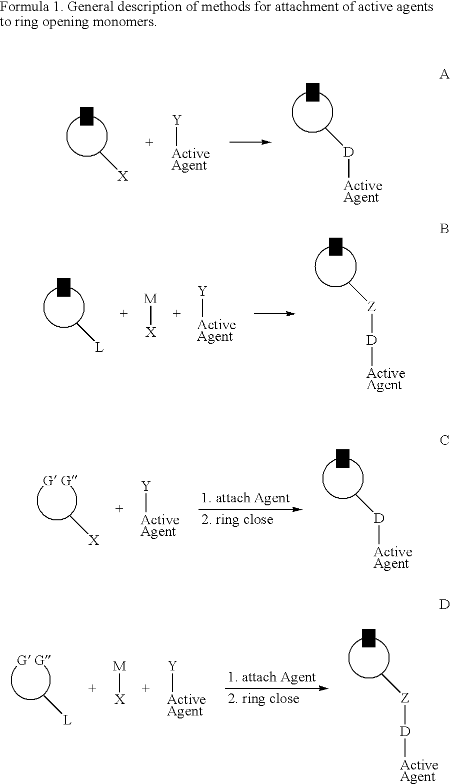

G is an atom or functional group in the ring structure that renders the cyclic monomer susceptible to ring-opening polymerization; G′ and G″ are atoms or functional groups that can undergo a ring-closing reaction; (X) and (Y) are complementary functional groups that form a degradable bond (D); and (L) and (M) are complementary functional groups that form a non-degradable bond (Z).

More specifically, G′ and G″ are atoms or functional groups that can combine via several alternative methods upon combination with the active agent. First, G′ and G″ can react with each other to form a new linkage (G). For example, an acid and alcohol can be combined to make an ester (lactone ring) or amine and an alcohol to make a lactam. Second, G′ and G″ can be modified through chemical transformation to react with each other. For example, ring closing polymerization can occur when G′ is a chloride and G″ is a carbonyl and the combination yields an epoxide ring. Third, G′ and G″ react with additional reagents. An example is provided below in Formula 10b when a diol with the attached active agent is reacted with ethylchloroformate to make the cyclic carbonate (G′ and G″ are both hydroxyls). Some other options for similar cyclic carbonate synthesis are shown in Formula 2.

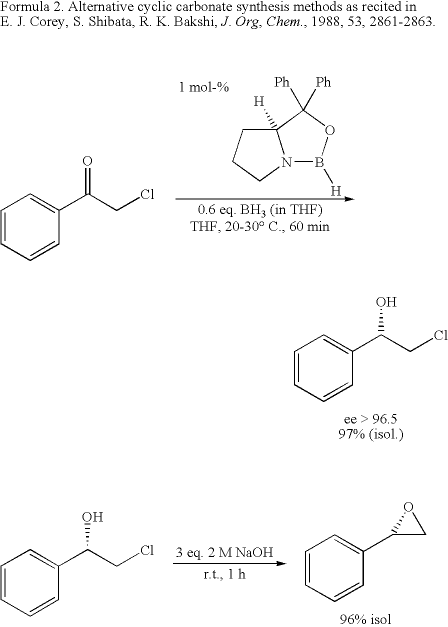

Ring-opening polymerization is defined as a form of addition polymerization, in which the terminal end of a polymer acts as a reactive center and further cyclic monomers join to form a larger polymer chain through propagation by opening of a cyclic structure. In many cases, these ring-opening polymerizations are controlled or “living-like” polymerizations. This allows advantages over conventional or non-controlled techniques such as: control over molecular weight (predetermined molecular masses can be achieved), low polydispersity index or narrow molecular weight distribution (most of the polymer chains are similar in length), the ability to create unique polymer architectures (such as blocks, stars, and grafts), and control of chain end groups.

Degradable covalent bonds are defined as covalent bonds that are broken via hydrolysis (reaction with water) under basic or acid conditions, metabolism, enzymatic degradation (by environmental and/or physiological enzymes), and other biological processes (such as those under physiological conditions in a vertebrate, such as a mammal) in less than 3 years.

Non-degradable covalent bonds are defined as those that are stable from hydrolysis (reaction with water) under basic or acid conditions, metabolism, enzymatic degradation (by environmental and/or physiological enzymes), and other biological processes (such as those under physiological conditions in a vertebrate, such as a mammal) for more than 3 years.

Examples of linking reactions that yield degradable bonds are as follows:
1. Alcohol+carboxylic acid, condensation reaction yields an ester bond
2. Alcohol+acid halide, condensation reaction yields an ester bond
3. Alcohol+acid anhydride, condensation reaction yields an ester bond
4. Alcohol+acid salts, condensation reaction yields an ester bond
5. Alcohol+isocyanate, addition reaction yields a urethane bond
6. Alcohol+ester, transesterification reaction yields a new ester bond
7. 2 carboxylic acids, dehydration yields an anhydride
8. Amine+isocyanate, addition reaction yields a urea bond
9. Amine+carboxylic acid, neutralization and dehydration reaction yields an amide bond
10. Amine+acid anhydride, substitution reaction yields an amide bond
11. Amine+acid halide, substitution reaction yields an amide bond
12. Amine+acid salts, reaction yields an amide bond
13. Amine+ester, reaction yields an amide bond
14. Amine+chloroformate, reaction yields a carbamate bond
15. Hydrazine+ketone or aldehyde, reaction yields a hydrozone bond

Several other groups can be used to attach the linker molecule to the ring opening monomer. These can be either degradable or non-degradable. Examples of these functional groups include: carboxylic acid, acid halides, acid salts, acid anhydride, hydroxyl, ester, amine, isocyanate, thiol, azide, nitrile, halide, unsaturated side chains, saturated side chains, aryl side chains, and heterocyclic side chains. The linker molecule can either be reacted first with the ring opening monomer (or precursor) or the active agent.

More generally, the combined conventional monomer and active agent and resulting polymer can be formed using the following process strategy.

### Cyclic Carbonate Starting Unit Synthesis and Drug-Modification

Cyclic carbonates can be synthesized from glycerol, a common trifunctional alcohol. In general, carbonates that can be used for this process can be represented by these structures:

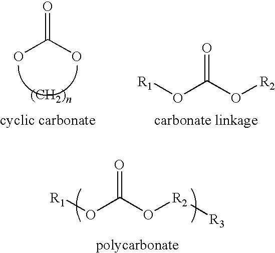

The proposed synthetic methods for creating the cyclic carbonate structures are shown below in Formula 3. In method A, the 1,2 diol can be selectively protected to form the 5 member cyclic carbonate by utilizing triphosgene and pyridine in DCM at low temperature and allowing the reaction to warm to ambient conditions. In method B, the two primary alcohols will be utilized to form the carbonate functional group while the secondary alcohol will remain available for covalent attachment of a drug molecule. A method using ethyl chloroformate as the carbonate-producing reagent is shown as Formula 3, method C. The reaction is carried out at mild temperatures (0-25° C.) with a stoichiometric amount of triethylamine as the acid scavenger, and six-member cyclic carbonates and obtained with approximately 60 percent yield. Ethyl chloroformate and other phosgene derivatives (phosgene, methyl chloroformate, di-, and triphosgene) have been used to produce cyclic carbonates from substituted 1,3-propane diols in relatively good yield (70 to 95 percent). The use of a stoichiometric excess of triethylamine improves the yield of cyclic carbonate. Once the cyclic carbonate has been formed, the remaining hydroxyl group is used for linking with the target drug via a condensation mechanism.

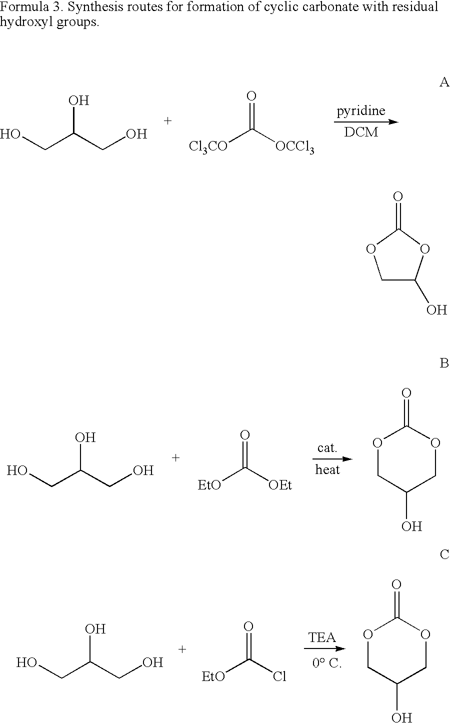

The residual hydroxyl group of the cyclic carbonate is modified with a drug molecule. The drug 2-(6-methoxynaphthalen-2-yl) propanoic acid (naproxen) is used in many experiments because it is readily available and is a well known anti-inflammatory. The attachment reaction is shown below in Formula 4. Coupling of a carboxylic acid with hydroxyl functionality is an extensively published reaction. Briefly, DCC coupling is afforded by dissolution of the alcohol in DCM at mild temperatures (0-25° C.) followed by the addition of DMAP, naproxen and N,N′-dicyclohexylcarbodiimide (DCC).

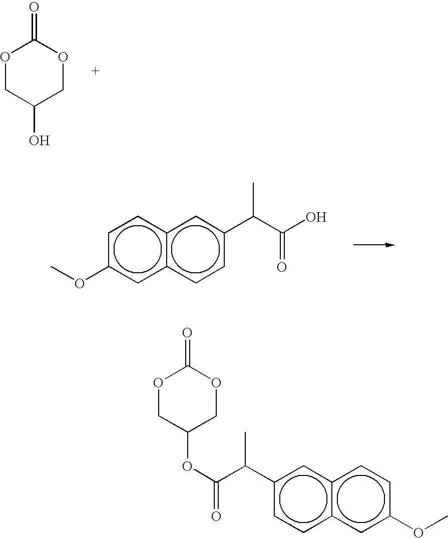

Formula 4. Drug-modification of a cyclic carbonate with naproxen.

Active agents with other functional groups can also be converted to ring opening monomers as long as they are reacted with a molecule containing a complementary functional group. For example, an amine functional active agent can be reacted with an acid functional molecule that can then undergo ring closing to yield the final ROP monomer. The reverse of the system described above can be conducted when an alcohol functional active agent is reacted with a carboxylic acid containing molecule, which can then undergo ring closing to yield the final ROP monomer. Examples of active agent residual functional groups include, but are not limited to: carboxylic acids, alcohols, amines, thiols, halides, unsaturated side chains, saturated side chains, aryl side chains, and heterocyclic side chains. Examples of functional group pairings include, but are not limited to: alcohols and isocyanates, amines and carboxylic acids, thiols and alkenes, acid salts and alkyl halides, and azides and alkynes.

### Epoxy Modification

Functional epoxides can be used with active agents with complimentary functional groups for forming the ring-opening monomer with epoxide functionality. For example, glycidol has both an epoxide and a terminal alcohol. Non-degradable linkages (must be used with a linker that would give a degradable linkage to the drug) include 3,4-Epoxy-1-butene, 2-Methyl-2-vinyloxirane, epichlorohydrin, epibromohydrin, 1,2-epoxy-5-hexene, glycidol propargyl ether, and methyl-2-methylglycidate. Degradable linkages include ethyl-2,3-epoxybutyrate, glycidyl methacrylate, 1,2,7,8-diepoxyoctane. The terminal alcohol group can be used to react by condensation with an acid functional active agent (such as naproxen). A practical example of this reaction is given in Example 3 of this specification.

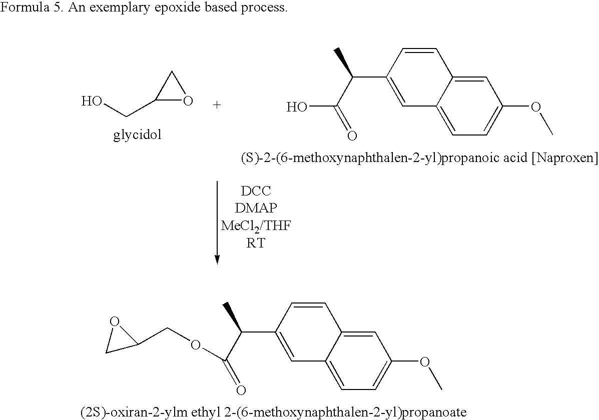

### Amine Modification

When using amino-functional materials, several ring closing reactions can be used to create lactams that can be polymerized by ring-opening polymerization. The general structure of a lactam is shown here:

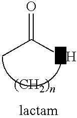

Generally, several linking reactions can be used to form active agent modified monomers. Examples of linking reactions that yield degradable bonds include the following.

1. Alcohol+Carboxylic Acid

This condensation reaction yields an ester bond when performed at room temperature to about 250° C., more typically from 70 to 200° C., and most preferably from 90-150° C. While not necessary, it is also preferred that the reaction be run in the presence of a catalyst, such as hydrochloric acid or sulfuric acid. A coupling agent such as a carbodiimide can also be used to facilitate the attachment of the alcohol and acid at lower temperature. The water of esterification can also be removed from the reaction mixture in order to drive the reaction to higher conversion

2. Alcohol+Acid Halide

This reaction yields an ester bond when performed at room temperature to about 230° C., more typically from 50 to 170° C., and most preferably from 70-120° C. While not necessary, it is also preferred that the reaction be run in the presence of an acid scavenger such as triethylamine. Removing the byproduct from the reaction mixture can drive the reaction to higher conversion.

3. Alcohol+Acid Anhydride

This condensation reaction yields an ester bond when performed at room temperature to about 230° C., more typically from 70 to 200° C., and most preferably from 80-150° C. While not necessary, it is also preferred that the reaction be run in the presence of a catalyst, such as hydrochloric acid or sulfuric acid. The byproduct of esterification can also be removed from the reaction mixture in order to drive the reaction to higher conversion.

4. Alcohol+Acid Salts

This condensation reaction yields an ester bond when performed at room temperature to about 250° C., more typically from 70 to 200° C., and most preferably from 80-150° C. While not necessary, it is also preferred that the reaction be run in the presence of a catalyst, such as hydrochloric acid or sulfuric acid. The byproduct of esterification can also be removed from the reaction mixture in order to drive the reaction to higher conversion.

5. Alcohol+Isocvanate

This addition reaction yields a urethane bond when performed at room temperature. Catalyst and/or can be added if needed to improve the reaction rate. The system should be kept free of water to avoid side reaction with the isocyanate.

6. Alcohol+Ester

This transesterification reaction yields a new ester bond when performed at room temperature to about 250° C., more typically from 110 to 220° C., and most preferably from 150-200° C. While not necessary, it is also preferred that the reaction be run in the presence of a catalyst, such as hydrochloric acid or sulfuric acid. The byproduct of transesterification can also be removed from the reaction mixture in order to drive the reaction to higher conversion.

7. 2 carboxylic Acids

This dehydration can be catalyzed using a variety of commercially available catalysts and/or the temperature should be raised to a temperature to allow for dehydration depending on the composition of the two acids.

8. Amine+Isocvanate

This addition reaction yields a urea bond when performed at room temperature or higher temperatures. Catalyst and/or can be added if needed to improve the reaction rate. The system should be kept free of water to avoid side reaction with the isocyanate.

9. Amine+Carboxylic Acid

This neutralization and dehydration reaction yields an amide bond. When the amine and carboxylic acid react upon mixing, the acid base neutralization forms ammonium carboxylate salts that can then be heated to greater than about 200° C. to dehydrate and form the amide bond.

10. Amine+Acid Anhydride

This substitution reaction yields an amide bond. The primary and secondary amines can react at low temperatures by nucleophilic acyl substitution to form amides generally in a mixed solvent system with water and an organic solvent.

11. Amine+Acid Halide

This substitution reaction yields an amide bond. The primary and secondary amines can react at low temperatures by nucleophilic acyl substitution to form amides generally in a mixed solvent system with water and an organic solvent.

12. Amine+Acid Salts

This reaction yields an amide bond. The amine and acid salts react through acid base neutralization to form ammonium carboxylate salts that can be heated to greater than about 200° C. to dehydrate and form the amide bond.

13. Amine+Ester

This reaction yields an amide bond and requires heating to from 50 to 250° C. and more preferably from 100 to 200° C. to form the bond.

14. Amine+Chloroformate

This reaction yields a carbamate bond and requires reaction at temperatures from -10 to 160° C. and more preferably from 0 to 50° C.

15. Hydrazine+Ketone or Aldehyde

This reaction yields a hydrazone bond. The reaction typically proceeds at temperatures ranging from 20 to 80° C.

### Active Agents

Several active agents can be selected for this process including drugs or other agents. Classes of drugs include cholinergics, adrenergics, serotonergics, anesthetics, hypnotics, antiseizure therapeutics, antipsychotics, anxiolytics, stimulants, opiods, analgesics, spasmolytics, cardiac glycosides, antianginals, antiarrhythmics, diuretics, angiotensin converting enzyme inhibitors, angiotensin converting enzyme antagonists, calcium blockers, central sympatholytics, peripheral sympatholytics, vasodilators, antihyperlipoproteinemics, cholesterol biosynthesis inhibitors, antithrombotics, thrombolytics, coagulants, plasma extenders, insulin, oral hypoglycemic agents, adrenocorticoids, estrogens, progestins, androgens, thyroid drugs, nonsteroidial anti-inflammatory agents, antihistamines, antiallergenic agents, antiulcer agents, antibiotics, antimicrobials, antiparasitics, antifungals, antimycobacterial agents, cancer chemotherapeutics, antivirals, protease inhibitors, gene therapeutics, antisense therapeutics, and selective estrogen receptor modulators.

Classes of other potential agents include sugars, carbohydrates, proteins, enzymes, RNA, DNA, pesticides, herbicides, anti-fouling agents, aromatic agents, detergents, sequestering agents, preservatives, anti-corrosion agents, and catalysts.

In addition to the covalently attached active agents, it may be desirable to incorporate one or more active agents that are not covalently attached. This can be done by several methods known in the art including but not limited to solvent blending with non-covalently attached active agent(s), melt blending with non-covalently attached active agent(s), co-extrusion with non-covalently attached active agent(s), coating of the polymer described in the invention with non-covalently attached active agent(s), or encapsulation of non-covalently attached active agent(s) with the polymer described in the invention.

The following is a more detailed list of possible active agents and the functional groups available for modification.

### Non-Steroidal Anti-Inflammatory Drugs (NSAIDs):

### Carboxylic Acids:

Aspirin
Diflunisal
Diclofenac
Aceclofenac
Acemetacin
Etodolac
Indometacin
Sulindac
Tolmetin
Ibuprofen
Carprofen
Fenbufen
Fenoprofen
Flurbiprofen
Ketoprofen
Ketorolac
Loxoprofen
Naproxen
Oxaprozin
Tiaprofenic acid
Suprofen
Mefenamic acid
Meclofenamic acid
Lumiracoxib

### Hydroxyl:

Oxyphenbutazone
Piroxicam
Lornoxicam
Meloxicam
Tenoxicam

### Steroidal Anti-Inflammatory Drugs:

### Hydroxyl:

Hydrocortisone
Prednisone
Prednisolone
Methylprednisolone
Dexamethasone
Betamethasone
Triamcinolone
Beclometasone
Fludrocortisone acetate
Aldosterone

### Chemotherapeutic Agents:

DNA alkylating Agents:
Melphalan (amine/acid)
Chlorambucil (acid)
Dacarbazine (amine)
Temozolomide (amine)
Streptozotocin (hydroxyl)

### Antimetabolites:

Methotrexate (acid/amine)
Pemetrexed (acid/amine)
Raltitrexed (acid)
Tioguanine (amine)
Fludarabine (amine/hydroxyl)
Pentostatin (hydroxyl)
Cladribine (amine/hydroxyl)
Floxuridine (hydroxyl)
Gemcitabine (amine/hydroxyl)

### Alkaloids:

Vincristine (hydroxyl)
Vinblastine (hydroxyl)
Vinorelbine (hydroxyl)
Vindesine (hydroxyl/amine)

### Topoisomerase Inhibitors:

Etoposide (hydroxyl)
Teniposide (hydroxyl)
Irinotecan (hydroxyl)
Topotecan (hydroxyl)

### Taxanes:

Paclitaxel (hydroxyl)
Docetaxel (hydroxyl)

### Anticoagulant:

Warfarin (hydroxyl)
Acenocoumarol (hydroxyl)
Phenprocoumon (hydroxyl)
Argatroban (acid/amine)
Ximelagatran (amine)

### Intermediate Composition Formation

Creation of PS-PIB-PS block copolymers. The structures in Formula 6 begin with the creation of a difunctional PIB block segment using QCP from the initiator 1,3-di(2-chloro-2-propyl)-5-tert-butylbenzene (bDCC). QCP of isobutylene and styrene will be carried out using well established procedures, employing TiC14 in a 60/40 (v/v) methylcyclohexane/methyl chloride cosolvent mixture within the temperature range -80 to -60° C. Real-time FTIR monitoring is used to determine the time of completion of the PIB block. Then, styrene is added sequentially to form PS-PIB-PS triblock. Conversion of the styrene is monitored using FTIR as described. The polymers are isolated by precipitation into methanol and dried in vacuo before site transformation of the end groups.

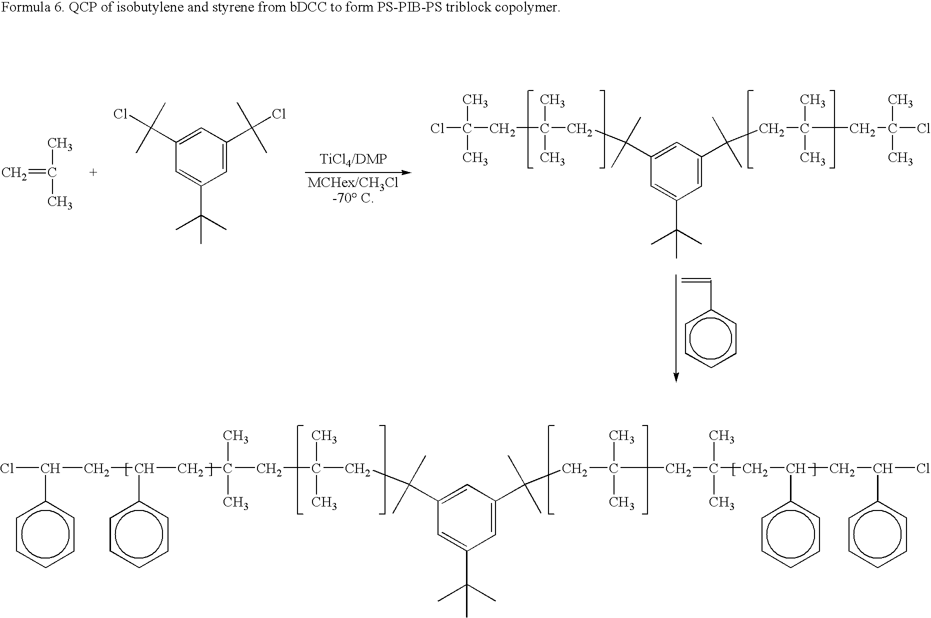

Several alternative monomers can be selected instead of the styrene and isobutylene based system. General classes of alternative monomers include the monomers of the following polymers: polyolefins, polyacrylates, polycarbonates, polyesters, polyamides, polyurethanes, polyethers, polyamideimides, polyaramide, polyarylate, polylactams, polylactones, polysiloxanes, polyesteramides, polyetherimides, polyetheretherketones, polyetherketones, polyethersulfones, polysulfides, polyketones, polyimides, polyols, polyphosphates, polyprryoles, polysilanes, polysilynes, polysilylenes, polysulfones, polycyclics, and natural polymers.

Site transformation/creation. For structures D and E of FIG. 1, the polymers obtained from QCP can be subjected to site transformation/creation to enable synthesis of the biodegradable poly(ester/carbonate) block. After quasiliving polymerization, the PS-PIB-PS polymers will have a styryl-chloride end-group configuration as shown in Formula 6. The polymers can then be subjected to reaction with allyltrimethylsilane, as demonstrated by Ivan, et al. and the allyl functional polymers will then undergo hydroboration oxidation reaction to yield a primary alcohol for ROP (Formula 7). Alternative methods to produce polymers with ROP initiating sites can also be utilized, such as using a functional capping agent during polymerization as described in U.S. Pat. No. 6,969,744 to Casey Stokes, et al.

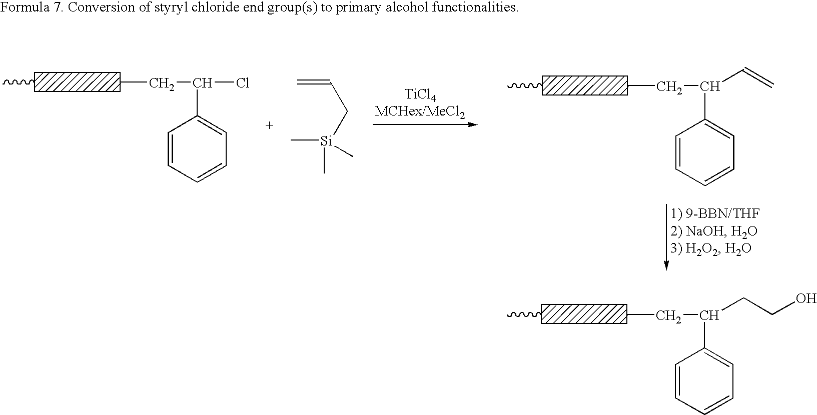

### Ring Opening Polymerization (ROP) of Active Agent-Modified Monomer or a Mixture Including the Active Agent-Modified Monomer.

The active agent-modified ring opening monomer can be polymerized either as a homopolymer or as one component of a mixed-monomer system. Because of the steric bulk added by the active agent attachment homopolymerization may not be possible and a comonomer may be necessary to form a polymer product containing the active agent-modified monomer units. Also, it may be desired to use more than one variety of active agent-modified monomer within one polymer system. The conditions for ROP will vary based on the composition of the monomer(s). Multiple monomers can be chosen to achieve co-, ter-, or higher order mixed-polymer products. Some options for comonomers include but are not limited to: cyclic ethers, cyclic esters (lactones), cyclic amides (lactams), N-carboxy-α-amino acid anhydrides, cyclic sulfides, siloxanes, and cyclic carbonates.

Second ring opening monomers, such as anionic- or insertion- ring opening monomers include cyclic carbonate, cyclic epoxide, lactam, lactone, lactide, anhydride, cyclic carbamate, cyclic phophoester, or siloxane. Specific examples of anionic- or insertion- ring opening monomers include: ethylene oxide, trimethylene oxide, oxepane, propylene oxide, epichlorohydrin, 3,3-bis-chloromethyloxetane, β-propriolactam, γ-butyrolactam, δ-valerolactam, ε-caprolactam, β-propriolactone, y-butyrolactone, δ-valerolactone, ε-caprolactone, L-lactide, D,L-lactide, glycolide, trimethylene carbonate, and octamethylcyclotetrasiloxane.

The ring opening polymerizations can be conducted using anionic or insertion-type mechanisms. There are several known initiators for these polymerization methods, however these systems are typically initiated by alcohols (which can vary in functionality) or alcohol/catalyst complexes (such as alkoxides). They can also be initiated by water, amines, porphyrins, or functional macroinitiators (oligomers, homopolymers, block copolymers, and other mixed-composition polymers). Some examples of macroinitiators include hydroxyl functional poly(ethylene glycol), hydroxyl functional polyisobutylene, and hydroxyl functional poly(styrene-b-isobutylene-b-styrene). The macroinitiators can be either degradable or non-degradable polymers. Suitable polymer macroinitiators include polystyrene, polybutylene, polyolefins, polyacrylates, polycarbonates, polyesters, polyamides, polyurethanes, polyethers, polyamideimides, polyaramide, polyarylate, polylactams, polylactones, polysiloxanes, polyesteramides, polyetherimides, polyetheretherketones, polyetherketones, polyethersulfones, polysulfides, polyketones, polyimides, polyols, polyphosphates, polyprryoles, polysilanes, polysilynes, polysilylenes, polysulfones, polycyclics, or natural polymers. Other suitable macroinitiators will be apparent to those of skill in the art and are to be considered within the scope of the present invention.

In an aspect, the block-polymer formed by reaction of the polymer described herein can further include at least one other polymer that can be polystyrene, polybutylene, polyolefins, polyacrylates, polycarbonates, polyesters, polyamides, polyurethanes, polyethers, polyamideimides, polyaramide, polyarylate, polylactams, polylactones, polysiloxanes, polyesteramides, polyetherimides, polyetheretherketones, polyetherketones, polyethersulfones, polysulfides, polyketones, polyimides, polyols, polyphosphates, polyprryoles, polysilanes, polysilynes, polysilylenes, polysulfones, polycyclics, or natural polymers. Other suitable polymer that can be used in embodiments of the present invention will be apparent to those of skill in the art and are to considered within the scope of the present invention.

The present invention can also include a block-polymer prepared by reacting the polymers described herein with one or more polymers comprising polystyrene, polybutylene, or polyethylene glycol.

The ring opening polymerizations can also be conducted where the more quickly polymerizing monomer is added to the polymerization mixture after initiation in order to allow time for the incorporation of the active agent modified monomer.

The catalyst system necessary for polymerization will depend on the monomer(s) selected with some examples being: stannous octoate, triethylaluminum, and other alkoxymetals. Other catalyst systems include N-heterocyclic carbenes, bifunctional thiourea-amines, “superbases”, enzymes, and other organic catalysts.

Generally speaking, ROP can be conducted in the bulk or in an appropriate solvent system. Toluene and tetrahydrofuran are known to be favorable ROP solvents.

The temperature of the reaction can vary from about 25 to 120° C. for solvent-based reactions and from about 25 to 250° C. for polymerizations in the bulk, with 90 to 150° C. preferred. The polymerizations do not necessarily require pressures above ambient pressure, but increased pressures can be used if the monomer mixture requires such (to keep the mixture in a non-gaseous state).

The following conditions for ROP of the cyclic ester and carbonate monomers are used: 130 ° C. toluene as solvent (only if necessary), and stannous octoate (Sn(Oct)2) as the catalyst (Formula 8). The monomers for copolymerization with the carbonates are D,L-lactide and glycolide; these have been previously used for biomedical applications, including drug-eluting stents. They have been shown to degrade in the body with rates adjustable by composition. Though the degradation products of PDLLA have been shown to elicit local inflammatory response when used as a bulk material for coronary stents, the existence of the polyester domains as a fraction of the stent material and the concomitant release of drugs from those domains should minimize, if not eliminate, such inflammatory response.

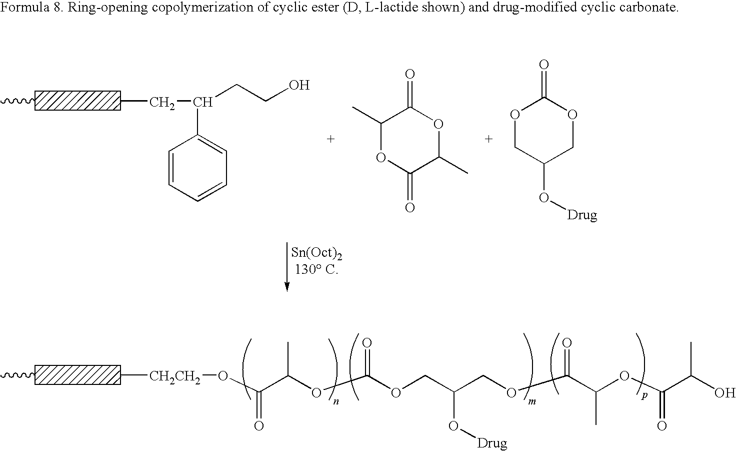

The molecular weight of the segments of the terpolymers can be varied in an attempt to achieve the desired 8-20 micron domain size for the degradable phases. The amount of drug-modified cyclic carbonate copolymerized into the degradable segment can also be varied. This allows control over the amount of drug loading in the terpolymer and can potentially affect the domain size by increasing the bulk of the degradable phase. Physical blends of the PS-PIB-PS triblocks with the poly (ester/carbonate) homopolymers can be made for comparison with the terpolymer results.

### Copolymerization

Many compositions can be used to produce degradable copolymers with these active agent modified ROP monomers, including those with more than one type of active agent-modified monomer. Almost any known ROP monomer can be used to copolymerize with the active agent ROP monomers. If the copolymerization of the ROP monomer and the active agent modified ROP monomer is very slow or impossible, a third monomer can be introduced. Experimentation shows that the copolymerization of naproxen modified TMC and L-lactide is very slow and did not readily show incorporation of the naproxen-TMC in the backbone of the copolymer. However, introduction of a third ROP monomer (glycolide) allows the copolymerization to proceed more quickly and incorporation of the TMC-Naproxen was confirmed. This illustrates the need to match the reactivity of the active agent modified ROP monomer with the reactivities of the desired comonomers. The compositions, however, are not limited to two or three comonomers.

The polymerization of these ROP monomers can be conducted with a variety of catalyst systems, some of the most common being stannous octoate, triethylaluminum, and other alkoxymetals. Other catalyst systems include N-heterocyclic carbenes, bifunctional thiourea-amines, “superbases”, enzymes, and other organic catalysts. The reactions can be conducted in the bulk or can be done in solution. The catalyst system, melt temperature of the monomers (for bulk systems), and reactivity of the monomers all dictate reaction temperatures that can range from 25 to 200° C., with typical temperatures from 80 to 180° C.

The reactivity of the comonomer must be “matched” to the reactivity of modified-monomers in order to get polymerization in a timely fashion (without other modifications to the polymerization method). In other words, if a monomer with low reactivity is used as the only comonomer with a modified-monomer that also has low reactivity, then the polymerization will be very slow - and possibly too slow to be practical. An alternative method involves using conditions in which the delivery rate of the more quickly polymerizing monomer is changed in order to allow for higher incorporation of the active agent modified monomer.

### Degradation of the Polymer or Mixed-Polymer to Release the Active Agent

The active agent can be released from the homopolymer or mixed-composition polymer containing the active agent-modified monomer units via degradation of the bond through which the active agent is attached to the back bone of the polymer. This degradation can occur via hydrolysis (reaction with water) under basic or acid conditions, metabolism, enzymatic degradation (by environmental and/or physiological enzymes), and other biological processes (such as those under physiological conditions in a vertebrate, such as a mammal). For ester degradation, the generation of acid functional groups during the degradation process provides an auto-catalytic effect by speeding further degradation of remaining ester bonds.

In general, release of the active agent involves the degradation of a biodegradable polymer into its subunits, or digestion of the polymer into smaller, non-polymeric subunits. Two different areas of biodegradation can occur: the cleavage of bonds in the polymer backbone that generally results in monomers and oligomers from the original polymer (Formula 9A); or the cleavage of a bond on the side chain or that connects a side chain to the polymer backbone (Formula 9B). This would not cause degradation of the polymer backbone, but would cause the release of the active agent from the polymer.

Release of the active agent is dependent on the stability of the degradable bond that is used to attach the agent to the polymer backbone and the degradation rate of the polymer backbone. Overall degradation of the polymer backbone can vary with polymer composition from about 3 weeks to greater than 3 years.

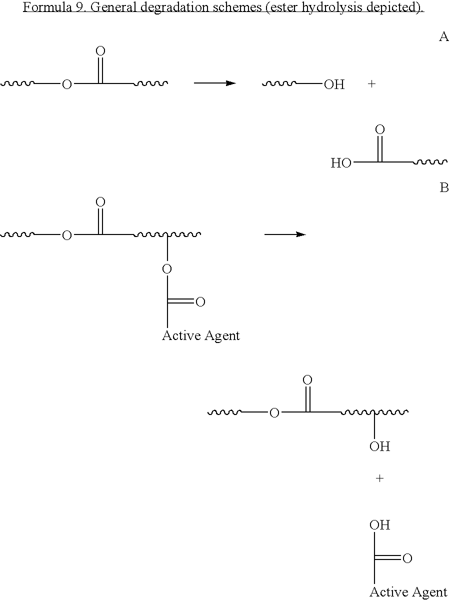

Generally, the degradable linkage will break and return the two original species (though now the monomer side will be the backbone of the polymer). For the polycarbonate and naproxen example given in the disclosure, the ester bond that attaches the naproxen to the backbone of the polymer is degradable by hydrolysis (reaction with water) and can break to form the acid form of the naproxen and a residual hydroxyl group on the carbonate linkage of the polymer backbone.

### Ring-Forming Polymerization of Active Agent-Modified Monomer or a Mixture Including the Active Agent-Modified Monomer.

The present invention also provides methods to form an active agent modified monomer comprising a compound including ring-forming complementary groups linked to an active agent via a degradable covalent linkage and methods to form a polymer or copolymer comprising an active agent modified monomer. The present invention provides methods to form an active agent modified monomer comprising combining a compound including a ring-forming complementary group with a first functional group (X) and an active agent with a second functional group (Y) to form an active agent modified monomer, wherein the first (X) and second (Y) functional groups are complementary functional groups that form a degradable linkage. The active agent modified monomer can also comprise a non-degradable linkage. In an aspect, the ring-forming complementary groups can include an alcohol and chloroformate, an alcohol and an acid, an amine and an alcohol, amine and an acid, acid halide and an alcohol, an acid halide and an amine, chloride and alcohol, two alcohols, or two acids.

In an aspect, the ring-forming complementary groups comprise an alcohol and chloroformate, an alcohol and an acid, an amine and an alcohol, amine and an acid, acid halide and an alcohol, an acid halide and an amine, chloride and alcohol, two alcohols, or two acids. Other suitable ring-forming complementary groups will be apparent to those of skill in the art and are to be considered within the scope of the present invention.

When various complementary groups are used, the ring-forming complementary groups can be closed using either direct condensation reactions or by addition of a ring-forming reagent, such as chloroformate or phosgene. As an example, when the ring-forming complementary groups are an amine and an alcohol, chloroformate or phosgene can be used to ring-close the structure. As another example, when two alcohol groups are used as the ring-forming complementary groups, chloroformate or phosgene can be used to ring-close the structure. Other suitable reactions or reagents can be used to close the ring-forming complementary groups. Such suitable reactions or reagents will be apparent to those of skill in the art and are to be considered within the scope of the present invention.

In another aspect, the ring-forming complementary groups form an epoxide, a cyclic carbonate, a lactone, an anhydride, a cyclic carbamate, or a lactam.

In another embodiment, the present invention provides methods of forming an active agent modified monomer comprising combining a compound including ring-forming complementary groups with a functional group (L), an active agent with a functional group (Y), and a linker with a functional group (X) and a functional group (M) to form an active agent modified monomer. In an aspect, the functional groups (X) and (Y) are complementary functional groups that form a degradable linkage and the functional groups (L) and (M) are complementary functional groups that form a stable or degradable linkage.

### Medical Devices

A material comprising the polymer can be formed into a medical device, as an active agent delivery vehicle, or used as a coating for a medical device. Use as a non-device, active agent delivery material includes injectible, insertable, or topical formulations as a standalone material or as a mixture with other active agents, solvents, or diluents. Preferred implantable or insertable medical devices for use in conjunction with the present invention include catheters (for example, renal or vascular catheters such as balloon catheters), guide wires, balloons, filters (e.g., vena cava filters), stents (including coronary vascular stents, cerebral, urethral, ureteral, biliary, tracheal, gastrointestinal and esophageal stents), stent grafts, cerebral aneurysm filler coils (including Guglilmi detachable coils and metal coils), vascular grafts, myocardial plugs, patches, pacemakers and pacemaker leads, heart valves, biopsy devices, or any coated substrate (which can comprise, for example, glass, metal, polymer, ceramic and combinations thereof) that is implanted or inserted into the body, either for procedural use or as an implant, and from which therapeutic agent is released.

The medical devices contemplated for use in connection with the present invention include drug delivery medical devices that are used for either systemic treatment or for the localized treatment of any mammalian tissue or organ. Non-limiting examples are tumors; interstitial spaces in joints; organs including but not limited to the heart, coronary and peripheral vascular system (referred to overall as “the vasculature”), lungs, trachea, esophagus, brain, liver, kidney, bladder, urethra and ureters, eye, intestines, stomach, pancreas, ovary, and prostate; skeletal muscle; smooth muscle; breast; cartilage; and bone.

One particularly preferred medical device for use in connection with the present invention is a vascular stent, which delivers therapeutic agent into the vasculature for the treatment of restenosis. As used herein, “treatment” refers to the prevention of a disease or condition, the reduction or elimination of symptoms associated with a disease or condition, or the substantial or complete elimination a disease or condition. Preferred subjects (i.e., patients) are mammalian subjects and more preferably human subjects.

Generally, examples of implantable or insertable medical device include catheters, guide wires, balloons, filters, stents, stent grafts, vascular grafts, vascular patches, and shunts. The implantable or insertable medical device can be adapted for implantation or insertion, for example, into the coronary vasculature, peripheral vascular system, esophagus, trachea, eye, colon, biliary tract, urinary tract, prostate or brain.

### Exemplary Procedure for Naproxen Polymer Formation

The structures in FIG. 1 can be achieved using a general synthetic strategy consisting of four steps, as follows:
1. Synthesis of cyclic carbonate with residual functionality and drug attachment.
2. Creation of a difunctional polyisobutylene (PIB) block using QCP initiated from bDCC followed by sequential polymerization of the polystyrene (PS) block.
3. Polymer isolation and preparation (site transformation/creation) for ROP (structures D and E).
4. Synthesis of poly (ester/carbonate) homopolymer or block segment by ROP and final isolation of polymer.

The following sections discuss these four steps in detail.

A carboxylic acid functional active agent (such as naproxen) is modified by esterification with an alcohol functional molecule as described in Formula 10a. The naproxen-modified alcohol is then ring-closed to yield a starting unit suitable for ring-opening polymerization as described in Formula 10b. The esterification of the carboxylic acid functional active agent and the alcohol functional molecule can be conducted under a range of temperatures (typically 70-200° C. or higher without catalyst, RT and higher with catalyst, and most frequently 80-150° C.) and typically in the presence of a catalyst, such as hydrochloric acid or sulfuric acid. A coupling agent such as a carbodiimide can also be used to facilitate the attachment of the alcohol and acid at lower temperature. The water of esterification can also be removed from the reaction mixture in order to drive the reaction to higher conversion.

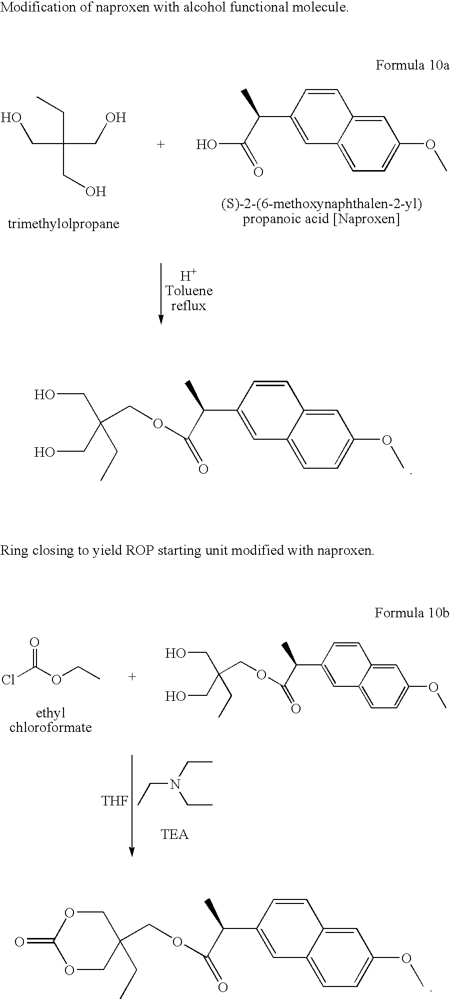

We have demonstrated the use of a carboxylic acid functional active agent (Naproxen) that is modified by esterification with an alcohol functional ring opening precursor molecule. The naproxen-modified alcohol is then ring-closed to yield a monomer suitable for ring-opening polymerization.

The following examples are included to demonstrate preferred embodiments of the invention. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments that are disclosed and still obtain a like or similar result without departing from the scope of the invention.

### EXAMPLES

For control experiments, a benzyl blocking agent can be used in place of a drug molecule.

### Example 1

A two step monomer synthesis beginning with trimethylol propane proceeds as follows.

Step 1: A 2000 mL one neck round bottom flask equipped with a Dean-Stark apparatus and a water-cooled reflux condenser was charged with 30.0 g naproxen (acidified form), 87.4 g trimethylol propane (5× excess to naproxen), 1000 mL Toluene, and 2 mL hydrochloric acid catalyst. The solution was brought to reflux temperature and allowed to stir for several hours until all of the water byproduct from esterification had been removed using the Dean-Stark apparatus. The solution was then cooled to room temperature and washed several times each with an aqueous saturated sodium bicarbonate (NaHCO3) solution, a 5 percent NaHCO3 solution, and a saturated brine (NaCl) solution. The organic layer was then dried by stirring over magnesium sulfate (MgSO4), the MgSO4 removed by filtration, and the organic solvent removed by rotary evaporation. The remaining product was recrystallized from diethyl ether, and the crystals were collected and dried in vacuo.

Step 2: A 2000 mL round bottom flask equipped with a dripping funnel, N2(g) purge, and an external ice water bath were added 20.0 g trimethylolpropane-modified naproxen (TMP-Naproxen, from Step 1 above), 800 mL tetrahydrofuran (THF), and 32.2 mL ethyl chloroformate (5.8× excess to TMP-Naproxen). The solution was allowed to cool to approximately 0° C. at which time 51.7 mL triethylamine (TEA, 6.4× excess to TMP-Naproxen) was added dropwise over at least 30 min. The reaction was removed from the ice bath and allowed to stir at room temperature for at least 2 h. The precipitated triethylamine hydrochloride was removed by filtration, and the filtrate was concentrated by rotary vacuum. The final product was recrystallized from THF/ether (½, v/v), and the crystals were collected and dried in vacuo.

### Example 2

The following is a representative procedure for the synthesis of 8 g of 25:25:50 D,L-lactide:glycolide:TMC-Naproxen with a molecular weight of 5000g/mol, performed inside the glove-box under an inert N2 atmosphere. A 100 mL two neck round bottom flask was first charged with 1.259 g of D,L-lactide, 1.005 g of glycolide, and 5.610 g of TMC-Naproxen. The flask equipped with a mechanical stirrer was then submerged into a silicone oil bath equilibrated at 60° C. The monomers were allowed to stir and completely dissolve. At this time, 1,4-butanediol (0.147 g) initiator was injected into the flask followed by 0.139g of stannous octoate (Sn(Oct)2), which serves as the catalyst. The reaction was allowed to run for four hours, with aliquots taken at defined intervals to monitor the reaction progress by GPC characterization. The final polymer was dissolved in chloroform and precipitated into methanol, isolated, and dried.

The synthetic preparation of the cyclic carbonate structures begin with a 1,3 diol, or the like, which is selectively protected to form the 5 or 6 member cyclic carbonate leaving the corresponding functional group available for covalent attachment of an active ingredient. An active ingredient can also be attached to the functional group prior to cyclization. Functional residual groups can include, but are not limited to, carboxylic acids, alcohols, amines, thiols, halides, unsaturated side chains, saturated side chains, aryl side chains, and heterocyclic side chains.

### Example 3

Additionally, a functionalized epoxide can be used in the monomer formation. An example 3 step synthesis beginning with glycidol proceeds as follows:

To a 500 mL one neck round bottomed flask were added 7.60 g naproxen (acidified form), 40 mL methylene chloride (MeCl2) and 25 mL THF. Once the naproxen had dissolved, 2.45 g glycidol was added and the entire system was purged with N₂(g). Then 5 g N,N′-diisopropylcarbodiimide (DIC) was added via syringe followed by 0.403g 4-dimethylaminopyridine (DMAP). The reaction mixture was allowed to stir overnight at room temperature. Water was added to the mixture, and the aqueous phase was extracted several times with MeCl₂. The combined organic extracts were washed with a saturated brine (NaCl) solution and dried over sodium sulfate (Na₂SO₄). The Na₂SO₄ was removed by filtration and the product isolated by rotary vacuum. The final product was dried in vacuo.

### Example 4

The following is a representative procedure for the synthesis of 4 g of 55:45 D,L-lactide:Epoxy-Naproxen with a molecular weight of 5000 g/mol, performed inside the glove-box under an inert N2 atmosphere. A 100 mL two neck round bottomed flask was first charged with 1.258 g of D,L-lactide and 2.651 g of Epoxy-Naproxen. The flask, equipped with a mechanical stirrer, was then submerged into a silicone oil bath equilibrated at 130° C. The monomers were allowed to stir and completely melt. At this time, 1,4-butanediol (0.072 g) initiator was then injected into the flask followed by 0.060 g of triethyl aluminum (AlEt3), which serves as the catalyst. The reaction was allowed to run for 24 hours, with aliquots taken at defined intervals to monitor the reaction progress by GPC characterization. The final polymer was dissolved in chloroform and precipitated into hexane, isolated, and dried.

The synthetic preparation of the functionalized epoxide structures begin with a glycidol, or the like, which is selectively attached to the active agent. Functional residual groups can include, but are not limited to, carboxylic acids, alcohols, amines, thiols, halides, unsaturated side chains, saturated side chains, aryl side chains, and heterocyclic side chains.

These materials can also be combined with desired pharmaceutical agents, which could be delivered over time. The desired pharmaceutical agents can possess functionality that complements attachment to the material (ie. Amine and hydroxyl functionality can be paired with a corresponding acid functionality, or the like, present in the active ingredient or vice versa). The delivery rate of these pharmaceutics can also be controlled by the composition of the polymers and the rate at which the degradable segments degrade.

### Comparative Example 5

A naproxen containing polymer was synthesized by the method as described in Example 2 using the following mixture of ring opening monomers: Lactide (2.3 g), Glycolide (1.8 g), and TMC-Naproxen (11.6 g). The resulting polymer was mixed with a 50:50 glycolide:lactide polymer containing zero naproxen (unmodified GLAC) as given in the table below. The 50:50 glycolide:lactide polymer containing zero covalently bound naproxen was also melt-mixed with free naproxen as indicated below. The two samples were shaped into disks and evaluated for drug release rate by immersion in a standard phosphate buffer solution at 7.4 pH. The samples were incubated and shaken at approximately 37° C. At the time intervals given in the chart below, the buffer solution was removed, analyzed for naproxen content by gas chromatography, and new buffer solution was added to the disks.

| | Naproxen- | Unmodified | |
|---|---|---|---|
| Sample ID | modified GLAC | GLAC | Free Naproxen |
| Bound naproxen | 0.3013 g | 0.9048 g | 0.0 g |
| Blended naproxen | 0.0 g | 1.3496 g | 0.1522 g |

### Example 6

The following is a representative procedure for the synthesis of 10 g of Glycolide:TMC-Naproxen block polymer. A 100 mL two neck round bottomed flask was first charged with 5.08 g of polymer macroinitiator, 3.75 g TMC-Naproxen monomer, 1.17 g glycolide, and 50 mL xylene. The flask, equipped with a magnetic stirrer, total condenser, and nitrogen purge, was then submerged into a silicone oil bath until reflux temperature was achieved. At this time, stannous octoate (0.051 g), which serves as the catalyst, was then injected into the flask. The reaction was allowed to run for approximately 17 hours. The final polymer was precipitated into methanol, isolated, and dried. Complete conversion of the naproxen monomer was not achieved, and the final naproxen content of the polymer was found to be approximately 1 wt %.

The preceding polymer was formed into approximately 10 mm×1 mm strips and submitted for animal study. The purpose of this study was to evaluate the local effects of a test article in direct contact with living skeletal muscle of the rabbit. The experimental design was as follows: 3 healthy adult New Zealand White rabbits were anesthetized, the test and control sites were prepared, and the test article and control article (USP High Density Polyethylene, Lot # HOF046) were implanted into the skeletal muscle. The control article was implanted into the left paraverebral muscle and the test article was implanted into the right paravertebral muscle of each rabbit. Five test article sites and five control article sites were implanted for each rabbit. The surgical sited were closed, and the animals were observed daily for 7 days (a week).

All tissues were fixed in 10% neutral buffered formalin. Hematoxylin and cosin (H&E) stained sections of the test and control implant sites were prepared from all animals. A veterinary pathologist microscopically evaluated the H&E stained tissue sections of each implant site. In comparison to the controls, the test articles showed a reduction in local inflammatory response (approximately 20%). In addition, the test articles were determined to be non-irritant with an irritation score of −0.2 (USP High Density Polyethylene control=0).

1. A method of forming an active agent modified monomer, comprising:
combining a ring opening cyclic monomer with a first functional group (X) and an active agent with a second functional group (Y) to form an active agent modified monomer,
wherein the first (X) and second (Y) functional groups are complementary functional groups that form a degradable linkage.

2. The method of claim 1 wherein the ring opening cyclic monomer is a cyclic carbonate, cyclic epoxide, lactam, lactone, lactide, anhydride, cyclic carbamate, cyclic phosphoester, or siloxane.

3. The method of claim 2 wherein the cyclic epoxide monomer is glycidol, ethyl-2,3-epoxybutyrate, glycidyl methacrylate, or 1,2,7,8-diepoxyoctane.

4. The method of claim 2 wherein the lactam monomer is 4-Oxo-2-azetidinecarboxylic acid, 4-Hydroxy-2-pyrrolidone, 5-(Hydroxymethyl)-2-pyrrolidinone, Pyroglutamic acid, Ethyl 2-oxo-3-piperidinecarboxylate, or alpha-Amino-epsilon-caprolactam.

5. The method of claim 2 wherein the cyclic carbonate monomer is 5-ethyl-5-(hydroxymethyl)-1,3-dioxan-2-one, 5-hydroxy-1,3 -dioxan-2-one, 4-hydroxy-1,3-dioxolan-2-one, 5-methyl-2-oxo-1,3-dioxane-5-carboxylic acid, or 5-ethyl-2-oxo-1,3-dioxane-5-carboxylic acid.

6. The method of claim 1 wherein the first (X) or the second (Y) functional group is independently an amine, aldehyde, ketone, chloroformate, hydrazine, alcohol, carboxylic acid, acid halide, acid anhydride, acid salt, isocyanate, or ester.

7. The method of claim 1, wherein the active agent modified monomer comprises a non-steroidal anti-inflammatory agents, chemotherapeutic agent, anticoagulant, cholinergics, adrenergics, serotonergics, anesthetics, hypnotics, antiseizure therapeutics, antipsychotics, anxiolytics, stimulants, opiods, analgesics, spasmolytics, cardiac glycosides, antianginals, antiarrhythmics, diuretics, angiotensin converting enzyme inhibitors, angiotensin converting enzyme antagonists, calcium blockers, central sympatholytics, peripheral sympatholytics, vasodilators, antihyperlipoproteinemics, cholesterol biosynthesis inhibitors, antithrombotics, thrombolytics, coagulants, plasma extenders, insulin, oral hypoglycemic agents, adrenocorticoids, estrogens, progestins, androgens, thyroid drugs, antihistamines, antiallergenic agents, antiulcer agents, antibiotics, antimicrobials, antiparasitics, antifungals, antimycobacterial agents, cancer chemotherapeutics, antivirals, protease inhibitors, gene therapeutics, antisense therapeutics, or selective estrogen receptor modulators, carbohydrates, proteins, enzymes, RNA, DNA, pesticides, herbicides, anti-fouling agents, aromatic agents, detergents, sequestering agents, preservatives, anti-corrosion agents, or catalysts.

8. The method of claim 1 wherein the first (X) and second (Y) functional groups react to form an ester, urethane, anhydride, carbonate, hydrazone, urea, or amide degradable linkage.

9. A method of forming an active agent modified monomer, comprising:
combining a compound comprising ring-forming complementary groups with a first functional group (X) and an active agent with a second functional group (Y) to form an active agent modified monomer, and
closing the ring-forming complementary groups using a direct condensation reaction or adding a ring-forming reagent to the ring-forming complementary groups;
wherein the first (X) and second (Y) functional groups are complementary functional
groups that form a degradable linkage.

10. The method of claim 9, wherein the ring-forming complementary groups comprise an alcohol and chloroformate, an alcohol and an acid, an amine and an alcohol, amine and acid, acid halide and an alcohol, an acid halide and an amine, chloride and alcohol, two alcohols, or two acids.

11. The method of claim 9, wherein the ring-forming complementary groups form an epoxide, a cyclic carbonate, a lactone, an anhydride, a cyclic carbamate, or a lactam.

12. A method of forming an active agent modified monomer, comprising
combining a ring opening monomer with a functional group (L), an active agent with a functional group (Y), and a linker with a functional group (X) and a functional group (M) to form an active agent modified monomer,
wherein the functional groups (X) and (Y) are complementary functional groups that form a degradable linkage and
wherein the functional groups (L) and (M) are complementary functional groups that form a stable or degradable linkage.

13. The method of claim 12 wherein the ring opening cyclic monomer is a cyclic carbonate, cyclic epoxide, lactam, lactone, lactide, anhydride, cyclic carbamate, cyclic phosphoester, or siloxane.

14. The method of claim 13 wherein the cycle epoxide is epichlorohydrin.

15. The method of claim 13 wherein the cyclic carbonate is

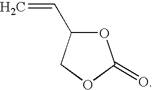

16. The method of claim 13 wherein the cyclic carbonate is

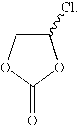

17. The method of claim 12 wherein the functional group (L) or (M) is an alkyne, alkene, alkyl halide, azide, thiol, or amine.

18. The method of claim 12 wherein the functional groups (L) and (M) react to form a thiolene, triazole, disulfide, or substituted amine.

19. The method of claim 12 wherein the functional group (X) or (Y) is an amine, alcohol, carboxylic acid, acid halide, acid anhydride, acid salt, isocyanate, aldehyde, ketone, chloroformate, hydrazine, or ester.

20. The method of claim 12 wherein the functional groups (X) and (Y) react to form an ester, urethane, carbonate, hydrazone, anhydride, urea, or amide bond.

21. The method of claim 12 wherein the active agent modified monomer is a non-steroidal anti-inflammatory agents, chemotherapeutic agent, anticoagulant, cholinergics, adrenergics, serotonergics, anesthetics, hypnotics, antiseizure therapeutics, antipsychotics, anxiolytics, stimulants, opiods, analgesics, spasmolytics, cardiac glycosides, antianginals, antiarrhythmics, diuretics, angiotensin converting enzyme inhibitors, angiotensin converting enzyme antagonists, calcium blockers, central sympatholytics, peripheral sympatholytics, vasodilators, antihyperlipoproteinemics, cholesterol biosynthesis inhibitors, antithrombotics, thrombolytics, coagulants, plasma extenders, insulin, oral hypoglycemic agents, adrenocorticoids, estrogens, progestins, androgens, thyroid drugs, antihistamines, antiallergenic agents, antiulcer agents, antibiotics, antimicrobials, antiparasitics, antifungals, antimycobacterial agents, cancer chemotherapeutics, antivirals, protease inhibitors, gene therapeutics, antisense therapeutics, or selective estrogen receptor modulators, carbohydrates, proteins, enzymes, RNA, DNA, pesticides, herbicides, anti-fouling agents, aromatic agents, detergents, sequestering agents, preservatives, anti-corrosion agents, or catalysts.

22. A method of forming an active agent modified monomer, comprising
combining a compound comprising ring-forming complementary groups with a functional group (L), an active agent with a functional group (Y), and a linker with a functional group (X) and a functional group (M) to form an active agent modified monomer,
closing the ring-forming complementary groups using a direct condensation reaction or adding a ring-forming reagent to the ring-forming complementary groups;
wherein the functional groups (X) and (Y) are complementary functional groups that form a degradable linkage and
wherein the functional groups (L) and (M) are complementary functional groups that form a stable or degradable linkage.

23. The method of claim 22, wherein the ring-forming complementary groups comprise an alcohol and chloroformate, an alcohol and an acid, an amine and an alcohol, amine and acid, acid halide and an alcohol, an acid halide and an amine, chloride and alcohol, two alcohols, or two acids.

24. The method of claim 22, wherein the ring forming complementary groups form an epoxide, a cyclic carbonate, a lactone, an anhydride, a cyclic carbamate, or a lactam.

25. An active agent modified monomer comprising a ring opening cyclic monomer linked to an active agent via a degradable covalent linkage.

26. The monomer of claim 25, wherein the ring opening cyclic monomer is a cyclic carbonate, cyclic epoxide, lactam, lactone, lactide, anhydride, cyclic carbamate, cyclic phosphonate, or siloxane.

27. The monomer of claim 26, wherein the cyclic epoxide is 3,4-Epoxy-1-butene, 2-Methyl-2-vinyloxirane, epichlorohydrin, epibromohydrin, 1,2-epoxy-5-hexene, glycidol propargyl ether, or methyl-2-methylglycidate.

28. The monomer of claim 26, wherein the lactam is bromocaprolactam, vinylcaprolactam, 5-chloromethyl-2-pyrrolidinone, 4-(2-propenyl)-2-pyrrolidinone, or 5-iodo-azocan-2-one.

29. The monomer of claim 26 wherein the cyclic carbonate is 5-ethyl-5-(hydroxymethyl)-1,3-dioxan-2-one, 5-hydroxy-1,3-dioxan-2-one, 4-hydroxy-1,3-dioxolan-2-one, 5-methyl-2-oxo-1,3-dioxane-5-carboxylic acid, or 5-ethyl-2-oxo-1,3-dioxane-5-carboxylic acid.

30. The monomer of claim 25 wherein the active agent is a non-steroidal anti-inflammatory agents, chemotherapeutic agent, anticoagulant, cholinergics, adrenergics, serotonergics, anesthetics, hypnotics, antiseizure therapeutics, antipsychotics, anxiolytics, stimulants, opiods, analgesics, spasmolytics, cardiac glycosides, antianginals, antiarrhythmics, diuretics, angiotensin converting enzyme inhibitors, angiotensin converting enzyme antagonists, calcium blockers, central sympatholytics, peripheral sympatholytics, vasodilators, antihyperlipoproteinemics, cholesterol biosynthesis inhibitors, antithrombotics, thrombolytics, coagulants, plasma extenders, insulin, oral hypoglycemic agents, adrenocorticoids, estrogens, progestins, androgens, thyroid drugs, antihistamines, antiallergenic agents, antiulcer agents, antibiotics, antimicrobials, antiparasitics, antifungals, antimycobacterial agents, cancer chemotherapeutics, antivirals, protease inhibitors, gene therapeutics, antisense therapeutics, or selective estrogen receptor modulators, carbohydrates, proteins, enzymes, RNA, DNA, pesticides, herbicides, anti-fouling agents, aromatic agents, detergents, sequestering agents, preservatives, anti-corrosion agents, or catalysts.

31. The monomer of claim 25, further comprising a non-degradable covalent linkage (Z) extending between the ring opening cyclic monomer and the degradable covalent linkage.

32. The monomer of claim 31, wherein the non-degradable covalent linkage (Z) comprises thiolene, triazole, disulfide, or substituted amine.

33. The monomer of claim 25 wherein the degradable covalent linkage is an ester, urethane, anhydride, carbonate, hydrazone, urea, or amide bond.

34. A polymer produced by the ring opening polymerization of the monomer of claim 25.

35. A polymer produced by the ring opening polymerization of the monomer of claim 25 and one or more other ring opening monomers.

36. The polymer of claim 35 wherein the one or more other ring opening monomers comprise cyclic carbonates, cyclic epoxides, lactams, lactones, lactides, anhydride, cyclic carbamate, cyclic phophoesters, siloxanes, or combinations thereof.

37. A block-polymer formed by ring opening polymerization of the monomer of claim 25 with at least one other ring opening monomer using a polymer macroinitator.

38. The polymer of claim 37, wherein the polymer macroinitiator comprises polystyrene, polybutylene, polyolefins, polyacrylates, polycarbonates, polyesters, polyamides, polyurethanes, polyethers, polyamideimides, polyaramide, polyarylate, polylactams, polylactones, polysiloxanes, polyesteramides, polyetherimides, polyetheretherketones, polyetherketones, polyethersulfones, polysulfides, polyketones, polyimides, polyols, polyphosphates, polypyrroles, polysilanes, polysilynes, polysilylenes, polysulfones, polycyclics, or natural polymers.

39. A block-polymer formed by a reaction of the polymer of claim 34 with at least one other polymer.

40. The polymer of claim 39, wherein the at least one other polymer comprises polystyrene, polybutylene, polyolefins, polyacrylates, polycarbonates, polyesters, polyamides, polyurethanes, polyethers, polyamideimides, polyaramide, polyarylate, polylactams, polylactones, polysiloxanes, polyesteramides, polyetherimides, polyetheretherketones, polyetherketones, polyethersulfones, polysulfides, polyketones, polyimides, polyols, polyphosphates, polypyrroles, polysilanes, polysilynes, polysilylenes, polysulfones, polycyclics, or natural polymers.

41. A block-polymer comprising a reaction product produced by reacting the polymer of claim 35 with one or more polymers comprising polystyrene, polybutylene, or polyethylene glycol.

42. A medical device comprising the polymer of claim 37.

43. The medical device of claim 42, wherein the device is a stent, a catheter, a guide wire, a balloon, a filter, a stent graft, a vascular graft, a vascular patch, and a shunt.

44. The medical device of claim 43, wherein the device is adapted for implantation or insertion into the coronary vasculature, peripheral vascular system, esophagus, trachea, colon, biliary tract, urinary tract, prostate, or brain.

45. A medical device coated by the polymer of claim 37.
